(19) United States
(12) Patent Application Publication
(10) Pub. No.: US 2007/0084727 A1
(43) Pub. Date: Apr. 19, 2007

(54) Coherent nonlinear chromatography and methods and devices thereof

(76) Inventor: Eric B. Cummings, Livermore, CA (US)

Correspondence Address: SMITH, GAMBRELL & RUSSELL (SNL), 1850 M STREET, NW, # 800, WASHINGTON, DC, 20036, US

(21) Appl. No.: 11/249,383

(22) Filed: Oct. 14, 2005

(51) Int. Cl. C07K 1/26; G01N 27/00

(52) U.S. Cl. 204/451; 204/601

(57) ABSTRACT

Disclosed herein are methods and devices for coherent nonlinear chromatography. As disclosed, the devices comprise microchannels having at least one perturber which produces a non-uniformity in a field spanning the width of the microchannel. The interaction of the field non-uniformity with a particle produces a secondary flow or particle motion component which competes with a primary flow. Depending on the interaction, the particle may be retained and redirected and therefore separated from other particles.

### ACKNOWLEDGEMENT OF GOVERNMENT SUPPORT

The present invention was made by employees of Sandia National Laboratories. The Government has certain rights in the invention.

### BACKGROUND OF THE INVENTION

1. Field of the Invention

The present invention generally relates to microfluidic and nanofluidic analysis of particles. In particular, the present invention relates assaying particles based on their mobilization characteristics in applied mobilization fields.

2. Description of the Related Art

Separation by size or mass is a fundamental analytical and preparative technique in biology, medicine, chemistry, and industry. Conventional methods include gel electrophoresis, field-flow fractionation, sedimentation and size exclusion chromatography.

A few microfluidic and nanofluidic devices for separation and analysis of biomolecules and compounds have been developed. U.S. Pat. No. 5,427,663 discloses separating nucleic acid molecules using electric fields through an array of posts as sieving matrices. Chou et al. disclose sorting nucleic acid molecules according to their diffusion coefficients using an electric field which propels the molecules through gaps formed by an asymmetric array of objects. See Chou et al. (1999) PNAS USA 96:13762. Han & Craighead disclose separations using entropic traps consisting of a series of many narrow constrictions (<100 nm) separated by wider and deeper regions (a few microns). See Han & Craighead (2000) Science 288:1026-1029. Huang et al. disclose a hexagonal array of posts which act as a sieving matrix in pulsed-field electrophoresis. See Huang et al. (2002) Nat. Biotechnol. 20:1048. U.S. Patent Publication 20040144651 discloses an array of obstacles wherein molecules are separated according to size.

All separation techniques work via a competition between mobilization and retention or dissipation transport mechanisms. In many conventional methods, the retention and drag mechanisms are stochastic. If these mechanisms can be modeled as a sequence of N discrete stochastic interactions, then the peak resolving power of these separations, at best scales as N^{-1/2}. At a constant average interaction rate therefore, the peak resolving power at best scales as t^{1/2}, where t is the duration of the separation process (this t^{1/2} scaling is quite general and is not restricted to retention and drag mechanisms that rely on discrete events). Each interaction is separated by a diffusive transport step through a mobile phase.

Moreover, incoherent retention and drag interactions essentially depend on diffusion or Brownian motion. For example, in conventional chromatography, a particle undergoing separation must diffuse between the stationary and mobile phases. This essential diffusive transport sets a limit on the rate of interactions a particle experiences. Fast, high-resolution separations require short diffusion times which in turn require small diffusion distances, small molecules, or both. A conventional approach to improving the separation resolving power for a given separation time is to reduce the diffusion distance by packing or otherwise porously filling a separation column or channel. If the mobile phase is moved by an applied pressure gradient, the small molecular diffusion distance comes at the cost of a correspondingly small viscous diffusion distance. Consequently the absolute pressure applied must be large, typically tens to many hundreds of atmospheres, to achieve a flow rate that is high enough that diffusive peak broadening does not reduce the resolving power. Even with such packings and high mobilization fields, these conventional diffusion-rate-limited separations perform relatively poorly for slowly diffusing particles of practical industrial, medical, and scientific interest, e.g., proteins and other biological macromolecules, polymers, and nanoparticles of natural, biological, and synthetic origin. Consequently, these prior art methods separate particles using diffusion mediated transport which detrimentally limits the sorting rate for large molecules.

Other prior art methods employ techniques in which particles come into direct contact with surfaces such that the particles experience steric effects. Such contact is detrimental, particularly for small particles, because steric effects increase sensitivity to surface fouling and increase the likelihood of surface fouling and create additional issues including complicated or high-precision fabrication requirements.

There is therefore a need for devices and methods for rapidly separating, concentrating, and assaying particles which are not diffusion-rate-limited or result in steric effects.

### SUMMARY OF THE INVENTION

The present invention provides devices and methods for rapidly separating, concentrating, and assaying particles which are not diffusion-rate-limited or result in steric effects. In particular, the present invention provides methods and devices for manipulating a particle in a fluid using coherent nonlinear chromatography (CNC).

In some embodiments, the present invention provides a method of manipulating an analyte in a fluid in a channel which comprises subjecting the analyte to a primary flow field and a secondary flow field produced by at least one field non-uniformity resulting from at least one perturber, with the proviso that where the perturber is a ridge obstacle or a valley obstacle, the primary flow field is not an electrokinetic field and the secondary flow field is not a dielectrophoretic field. In some embodiments, the primary flow is an electrokinetic flow or a hydrodynamic flow. The perturber is an obstacle, a patch, or a projection. In preferred embodiments, the perturber is elongated. In some embodiments, the secondary flow field is an electrophoretic field, a dielectrophoretic field, a magnetophoretic field, an electrostriction field, a photophoretic field, a thermophoretic field, an entropic field, an acoustical field, or a chemical field. In some embodiments, the secondary flow field is produced by a plurality of field non-uniformities in a coherent array. In some embodiments, the methods include placing the perturber at an angle that is substantially perpendicular to the primary flow field. In some embodiments, the methods include placing the perturber at an angle to the perpendicular of the primary flow field. In some embodiments, the methods include placing the field non-uniformity at an angle that is substantially perpendicular to the primary flow field. In some embodiments, the methods include placing the field non-uniformity at an angle to the perpendicular of the primary flow field.

In some embodiments, the present invention provides an assay for an analyte in a fluid which comprises manipulating the analyte using the methods or devices described herein and observing or detecting the secondary flow of the analyte. In some embodiments, the assay further comprises comparing the secondary flow of the analyte with a control. In some embodiments, the control is the primary flow, the movement of a given or known analyte, and the like.

In some embodiments, the present invention provides a microfluidic device comprising at least one perturber capable of producing at least one field non-uniformity in a primary flow field in a channel, with the proviso that where the perturber is a ridge obstacle or a valley obstacle, the primary flow field is not an electrokinetic field and the secondary flow field produced by the field non-uniformity is not a dielectrophoretic field. The perturber is an obstacle, a patch, or a projection. In preferred embodiments, the perturber is elongated. In some embodiments, the device comprises a plurality of perturbers, which may be the same or different. In some embodiments, the device comprises two or more domains of perturbers which may be in series or parallel. The domains may have structure which separates them such as a ridge, a channel, a binning channel, a valve, or the like. In some embodiments, the perturber is at an angle that is substantially perpendicular to the primary flow field. In some embodiments, the perturber is at an angle to the perpendicular of the primary flow field. In some embodiments, the field non-uniformity is at an angle that is substantially perpendicular to the primary flow field. In some embodiments, the field non-uniformity is at an angle to the perpendicular of the primary flow field.

Both the foregoing general description and the following detailed description are exemplary and explanatory only and are intended to provide further explanation of the invention as claimed. The accompanying drawings are included to provide a further understanding of the invention and are incorporated in and constitute part of this specification, illustrate several embodiments of the invention, and together with the description serve to explain the principles of the invention.

### DESCRIPTION OF THE DRAWINGS

This invention is further understood by reference to the drawings wherein:

FIGS. 1A-1C show examples of how projection perturbers produce spatially non-uniform fields in channels. The channel walls are represented by the two parallel parallelograms (the bottom view is partially obscured). The solid arrows indicate the primary flow. The dotted arrows indicate the direction of the source of the applied field and the gray straight lines indicate an unperturbed field. The gray waves indicate a spatially non-uniform field.

FIG. 1A shows a diagram of a projection in which a spatially non-uniform field is prepared outside the channel and extends through the channel.

FIG. 1B shows a diagram of a projection in which a spatially non-uniform field is formed in a channel via interference, standing, or traveling waves inside a channel.

FIG. 1C shows a diagram of a static or moving interference pattern in a field projected into a channel by propagating two or more coherent acoustic or optical waves into a channel.

FIGS. 2A and 2B show examples of how obstacle perturbers modulate applied fields. The channel walls are represented by the two parallel parallelograms (the bottom view is partially obscured). The solid arrows indicate the primary flow. The dotted arrows indicate the direction of the source of the applied field and the straight gray lines indicate the unperturbed field. The gray waves indicate the spatially non-uniform field produced by the obstacles. The shape on the underside of the top parallelogram represents elongated obstacles on the inside channel surface.

FIG. 2A shows how obstacles can directly perturb an externally applied field having a component normal to the flow channel.

FIG. 2B shows how obstacles can directly perturb an axial applied field or field that propagates through or is associated with the fluid within the channel.

FIGS. 3A-3C show schematic diagrams of how patch perturbers modulate applied fields and directly drive non-uniform fields. The channel walls are represented by the two parallel parallelograms (the bottom view is partially obscured). The solid arrows indicate the primary flow. The dotted arrows indicate the direction of the source of the applied field and the straight gray lines indicate the unperturbed field. The gray waves indicate the spatially non-uniform field produced by the patches. The solid black stripes represent elongated patch perturbers.

FIG. 3A shows how patches can perturb the propagation of an external applied field into the flow channel.

FIG. 3B shows how patches can perturb the propagation of an axial or fluid-associated field.

FIG. 3C shows how patches can directly drive a spatially non-uniform field or can perturb intrinsic or spontaneously forming fields within a channel.

FIGS. 4A-4G show cross-sections of particle potential energy perturbations produced by examples of perturbers according to the present invention. The fringes are the contours of constant potential energy and the overlaid lines represent unperturbed streamlines. The perturbation force and terminal velocity associated with these potentials are normal to the fringes.

FIG. 4A shows the electrostrictive or photophoretic potential energy resulting from an externally applied sinusoidal optical intensity distribution or interference pattern.

FIG. 4B shows the dielectrophoretic potential energy resulting from electric-field non-uniformities produced by insulating obstacles.

FIG. 4C shows entropic potential energy resulting from flow-induced particle distortion produced by obstacles

FIG. 4D shows the electrostatic potential energy resulting from periodic electric double layer overlap over obstacles.

FIG. 4E shows the electrostatic potential energy in a channel having nearly overlapped electric double layers resulting from patches creating periodically poled charges at the bottom surface.

FIG. 4F shows the electrostatic potential energy in a channel resulting from patches having periodically patterned conductivity at the bottom surface.

FIG. 4G shows the electrostatic potential energy resulting from a conductive patch and obstacle.

FIGS. 5A-5H show cross-sections of examples of ridge and valley obstacles.

The right panel of each figure shows the cross-sections of the particle potential energy perturbations produced by non-uniform fields. The fringes are contours of constant potential energy.

FIG. 5A generally represents a cross-section of a ridge formed using a high aspect ratio etching procedure.

FIG. 5B is the valley corresponding to the ridge of FIG. 5A.

FIG. 5C generally represents a cross-section of a ridge formed using an anisotropic low-aspect ratio fabrication technique.

FIG. 5D is the valley corresponding to the ridge of FIG. 5C.

FIG. 5E generally represents a cross-section of a ridge formed using isotropic etching techniques.

FIG. 5F is the valley corresponding to the ridge of FIG. 5E.

FIG. 5G generally represents a cross-section of a ridge formed using molding or stamping.

FIG. 5H is the valley corresponding to the ridge of FIG. 5G.

FIGS. 6A-6E represent a simulation of a time-of-flight separation using a coherent array according to the present invention. FIG. 6A is at time 0 wherein a fluid comprising three different particles (differentially interact with the field non-uniformities created by the ridges) are introduced to the coherent array. FIGS. 6B-6E are consecutive periods of time.

FIG. 7A is a numerical simulation showing the tracks of particles that interact with the non-uniform field such that they are deflected by an angle that depends on the strength of the particle-non-uniform-field interaction.

FIG. 7B is a numerical simulation showing the tracks of particles that interact strongly enough with the non-uniform fields to be inhibited from crossing the perturbers and propagate substantially along the length of the perturbers.

FIG. 8 is a diagram of an example of a multiple-domain continuous particle spectrometer according to the present invention wherein particles are deflected but not inhibited by the interactions with the non-uniform fields.

FIGS. 9A-9C are diagrams of particle motion past elongated perturbers. Particles for whom the interaction with the perturber is attractive dwell at the downstream side of a perturber, while repelled particles dwell at the upstream side.

FIG. 9A is a diagram of particle motion past elongated perturbers where the secondary flow is uncoupled or only weakly coupled to the primary flow, i.e., n<1, the ability of the perturber to inhibit transport past the perturber decreases with increasing angle between the axis of the perturber and the primary flow. Weakly interacting particles propagate a short distance along such a curved perturber while more strongly interacting particles propagate further along the perturber before escaping the interaction. The action of a ridge having such curvature is thus that of a continuous transverse particle spectrometer base upon the magnitude of the interaction with the perturber.

FIG. 9B is a diagram of particle motion past elongated perturbers where the secondary flow normal to the perturber scales linearly with the primary flow, there is no angle dependence of the inhibition threshold. Particles that are not inhibited from crossing the perturber can nevertheless be continuously sorted transversely via the magnitude of the weak retention effect produced by the interactions, which collectively cause particle paths to follow a trajectory whose angle with the primary flow increases with the amount of interaction, an effect that occurs for all values of n.

FIG. 9C is a diagram of particle motion past elongated perturbers where the secondary flow is strongly coupled to the primary flow, i.e., n>1, the angular dependence of particle inhibition is reversed from that in A. The perturber must therefore be curved in the opposite direction from A to achieve a spectrometer function based upon the inhibition threshold.

FIG. 10A is a diagram of a multiple-domain continuous-flow spectrometer according to the present invention in which n>1 and particles are substantially inhibited by their interactions with the non-uniform fields, depending on the local angle of the perturber with the primary flow.

FIG. 10B is a numerical simulation of polydisperse particle transport with diffusion in the spectrometer of FIG. 10A. Two particle classes are highlighted black (more strongly interacting) and white (less strongly interacting).

FIG. 11 is an example diagram of an asymmetric perturber array that supports ratcheting separations in which the direction of the primary flow field is periodically reversed.

### DETAILED DESCRIPTION OF THE INVENTION

The present invention provides methods and devices for employing coherent particle scattering (CPS) to separate particles based on their volume interactions with competing mobilization fields. The application of CPS to particle sorting and separation is referred to as “coherent nonlinear chromatography” (CNC).

Coherent particle scattering (CPS) is transport produced by deterministic particle interactions with field non-uniformities. These interactions include electrostatic, dielectrophoretic, magnetic, electromagnetic, optical, inertial, hydrodynamic, and mechanical interactions that occur through a significant fraction of the cross-section or “bulk” of the flow channel. These “volume” interactions contrast with “surface” interactions like the hydrophobic interactions used in chromatography, and direct physical entanglement and mechanical-contact-based sieving techniques. Because the scattering events are deterministic, the peak resolving power of separations based on CPS can in principle scale linearly with time when they are driven coherently, e.g., by spatially periodic repetition of the perturbation, requiring far fewer interactions than conventional techniques to produce a desired separation.

CPS does not require pore- or channel-scale diffusion for each interaction and is thus not inherently diffusion-rate limited. At most, molecular-scale diffusion time is needed for interactions that involve thermal motion to relax or equilibrate from a perturbation in the physical arrangement or excited energy state, e.g., thermal relaxation of particle conformation following a mechanical distortion. Electrostatic, magnetic, electromagnetic, dielectrophoretic, and hydrodynamic interactions do not explicitly employ thermal relaxation or equilibration or diffusion. These interactions are rate dependent because of other finite-rate relaxation mechanisms, e.g., polarization relaxation in dielectrophoresis. Because the magnitude of CPS depends on finite relaxation rates, CNC can actually be used to sort particles by their perturbation relaxation rates, i.e., time required for a particle to return to an equilibrium state following a perturbation. If relaxation rates are sufficiently high, CNC can be performed in an ultrafast manner, e.g., inertial large protein separations can theoretically be conducted in the ten-millisecond time scale instead of the kilosecond time scale typical for conventional chromatography. On the other hand, if the relevant relaxation mechanism, i.e., the physical process by which a perturbed particle re-equilibrates, is relatively slow, the linear scaling of resolving power with the number of interactions N, as opposed to N^{1/2} for conventional chromatography, in principle, allows the separation to proceed as fast as is physically possible for a given perturbation and relaxation mechanism.

In some cases, channel-scale diffusion, i.e., diffusion through the channel depth, span, or other characteristic geometrical length scale, can improve the performance of CNC separations by averaging out streamline-dependent phenomena that produce hydrodynamic dispersion. A preferable alternative to diffusion for reducing hydrodynamic dispersion is focusing or concentration of the target particles toward preferred streamlines of the primary flow. An effect of the secondary flow, this primary-flow-transverse focusing forces particles into narrow streams that substantially follow particular flow streamlines. Such transverse focusing can significantly reduce the amount of diffusion needed to average over streamline-to-streamline variations in flight-times in CNC devices.

As provided herein, the present invention provides methods and devices for separating or selectively concentrating particles in a fluid using coherent nonlinear chromatography (CNC) in a channel, preferably a microchannel. As used herein, “channel” refers to a structure wherein a fluid may flow. A channel may be a capillary, a conduit, a strip of hydrophilic pattern on an otherwise hydrophobic surface wherein aqueous fluids are confined, and the like. As used herein, “microfluidic” refers to a system or device having one or more fluidic channels, conduits or chambers that are generally fabricated at the millimeter to nanometer scale. Thus, the “microfluidic channels” or alternatively referred to herein as “microchannels” of the present invention generally have cross-sectional dimensions ranging from about 10 nm to about 1 mm.

As used herein, a “fluid” refers to a substance that tends to flow and to conform to the outline of a container such as a liquid or a gas. Fluids include saliva, mucus, blood, plasma, urine, bile, breast milk, semen, tears, water, liquid beverages, cooking oils, cleaning solvents, hydrocarbon oils, fluorocarbon oils, ionic fluids, air, and the like. Fluids can also exist in a thermodynamic state near the critical point, as in supercritical fluids. If one desires to test a solid sample for a given analyte according to the present invention, the solid sample may be made into a fluid sample using methods known in the art. For example, a solid sample may be dissolved in an aqueous solution, ground up or liquefied, dispersed in a liquid medium, melted, digested, and the like. Alternatively, the surface of the solid sample may be tested by washing the surface with a solution such as water or a buffer and then testing the solution for the presence of the given analyte. A fluid may be a polydisperse fluid, i.e. a fluid comprising a variety of particles having different properties, particles having multiple properties, or both.

As used herein, “analyte” is used interchangeably with “particle” to refer to a particle that may be natural or synthetic chemicals and biological entities. Chemicals and biological entities (biomolecules) include industrial polymers, powders, latexes, emulsions, colloids, environmental pollutants, pesticides, insecticides, drugs such as cocaine and antibiotics, magnetic particles, high-magnetic-permeability particles, metal ions, metal ion complexes, inorganic ions, inorganic ion complexes, organometallic compounds, metals including aluminum, arsenic, cadmium, chromium, selenium, cobalt, copper, lead, silver, nickel, and mercury, and the like, amino acids, peptides, proteins, glycoproteins, nucleotides, nucleic acid molecules, carbohydrates, lipids, lectins, cells, viruses, viral particles, bacteria, organelles, spores, protozoa, yeasts, molds, fungi, pollens, diatoms, toxins, biotoxins, hormones, steroids, immunoglobulins, antibodies, supermolecular assemblies, ligands, catalytic particles, zeolites, and the like, biological and chemical warfare agents, agents used in explosives, and the like.

As used herein, “separating” is used interchangeably with “sorting”, “collecting,” “concentrating,” “filtering,” “assaying,” “detecting” “measuring, ” “monitoring,” and “analyzing”. As provided herein, particles are separated by CNC, a process in which particles are made to depart from a primary flow via a secondary flow either longitudinally, resulting in a modified time-of-flight of the particles through a channel, or transversely, resulting in a modified spatial distribution of particles in a channel, or a combination of longitudinal and transverse processes.

Specifically, the present invention provides methods and devices which separate particles based on one or more characteristics by competing a primary flow field (primary mobilization field) with a secondary flow field in a channel. As used herein, “mobilization field” refers to any force field that influences a particle to pass through a channel or region of a channel. Mobilization fields include hydrodynamic flow fields produced by pressure differences, gravity, linear or centripetal acceleration, electrokinetic flow fields, electroosmotic flow fields, magnetophoretic and thermophoretic flow fields, electric fields, optical fields, centrifugal fields, gravitational fields, combinations thereof, and the like.

The primary flow of the particles is the motion of particles (flow) resulting from a primary flow force exerted by the primary flow field. Primary flow is generally in a direction that is substantially parallel to the boundaries of a microchannel. As used herein, a “primary flow force” is the force on a particle that makes it follow the primary flow. Two classes of primary flow are considered herein, “hydrodynamic flows” which, as used herein, include pressure-gradient-, capillarity-, inertia-, gravity, centripetal-acceleration-, chemical reaction-, and magnetically-driven flows and the like and “electrokinetic” flows which are the superposition of electrophoretic and electroosmotic flows. The electrokinetic flows considered herein can have substantial electrophoretic and electroosmotic components or have substantially dominant electrophoretic or electroosmotic components either by the choice of channel boundaries, fluid composition, surface treatments, surfactants, dynamic coatings, gels, and the like, including those known in the art.

The secondary flow field retains or redirects particles from the primary flow. The secondary flow field is produced by mechanical interactions, inertial interactions, entropic interactions, electrostatic interactions, magnetic interactions, electromagnetic interactions, optical interactions, chemical interactions, or a combination thereof, with at least one field non-uniformity projected or produced from patterns in the channel.

Secondary flow generally refers to the motion of a particle resulting from its interactions with forces produced by the secondary flow field and the “secondary flow force” refers to the force that produces the secondary flow. The secondary flow in the present invention is produced by interactions that occur substantially throughout the cross-section of the primary flow at a region or regions in the channel and are referred to as “volume interactions”. As used herein, “volume interaction” refers to any force on a particle that a particle can experience substantially throughout the cross-section of a flow channel. Thus, “secondary flow” is the particle motion produced by volume interactions resulting from at least one spatially non-uniform field within the flow channel

Volume interactions are different from “surface interactions”, which occur only when a particle is in immediate proximity to a channel boundary. As used herein, “surface interaction” refers to any force on a particle that a particle only experiences in immediate proximity to a boundary of the flow channel. Such surface interactions include Van Der Waals and other “adhesive” forces, electrostatic forces within a Debye layer that is thin compared other channel dimensions, steric effects and mechanical reaction forces produced when particles are forced onto or collide with a surface, and the like. These surface interactions are not employed to produce a secondary flow according to the present invention.

As used herein, a “field non-uniformity” refers to a spatial gradient in a field. As provided herein, a field non-uniformity is produced within the channel by a perturber. As used herein, a “perturber” distorts an applied flow, an electric field, or the action of an area or region in a channel which produces gradients in an intrinsic field, e.g., the electric field within the Debye layer. A perturber may be a projection. As provided herein, a “projection” is a disruption projected into a uniform field to produce a field non-uniformity. Examples of disruptions include optical interference patterns, temperature fields, magnetic fields, acoustic energy fields, and the like. These disruptions may modulate particle motion directly, e.g., by acoustic streaming or electrostriction, or modulate the behavior of the particle in the primary flow, e.g., by modifying surface charge density and thereby mobility in an electrokinetic primary flow. Moreover, all the force for the secondary flow can be generated from the action of a non-uniform primary flow, e.g., dielectrophoresis in a high-field electrokinetic flow or inertia in a high-speed hydrodynamic flow. The degree of coupling between the primary and secondary flows can be quantified by a simple exponent, n, which is an important consideration in the design of CNC devices.

A perturber may be an obstacle. As provided herein, an “obstacle” is a protrusion or a cavity in a surface of a channel. For example, a ridge is an elongated protrusion and a valley is an elongated cavity in a surface of a channel. As used herein, “elongated” refers to an object that has a length that is greater than its width, but is not necessarily straight. As used herein, a “post” refers to an arbitrarily shaped object that spans the channel either straight or at an incline with respect to the channel boundaries. As used herein, a “hole” refers to an opening in the channel through which particles, fluid or both may pass. In preferred embodiments, the perturbers are elongated.

Obstacles of the present invention may be made from the same material as the material defining the microfluidic channel in which the obstacle is located or the obstacles may be made of a different material deposited or adhered to walls of the microchannels using methods known in the art. Suitable materials for the pertubers include materials that are insulative, conductive, semi-conductive, or a combination thereof.

As used herein, the word “conductivity” is used to describe the ease of flow of both conduction and displacement current. It is often mathematically described as a complex number that varies with the frequency of the applied electric field. Similarly, “conduction” is used to describe both conventional conduction and conduction of displacement currents. As used herein, “insulative” and “conductive” refers to the relative conductivity of the described item with respect to the fluid. Insulative materials have relatively low conductivity and include plastics, epoxies, photoresists, polymers, silicon, silica, quartz, glass, controlled pore glass, carbon, and the like, and combinations thereof. Preferred insulative materials include thermoplastic polymers such as nylon, polypropylene, polyester, polycarbonate and the like. Conductive materials, in comparison, have relatively high conductivity. Conductive materials include bulk, sputtered, and plated metals and semiconductors, carbon nanotubes, and the like.

A perturber may be flush with the surface of the channel and is herein referred to as a “patch.” A patch is region having at least one property that is different from the adjacent regions of the surface of the channel. Such properties include surface charge, conductivity, transparency, absorptivity, and dopant concentration and type (which may be produced statically during fabrication or dynamically during device operation), and the like. Suitable materials for the patches include materials that are, relative to the adjacent surface, insulative, conductive, photo-conductive, photo-cleavable, semi-conductive, charged, neutral, high dielectric constant, low dielectric constant, positive Zeta potential, negative Zeta potential, or a combination thereof.

One skilled in the art may readily combine different perturbers, arrays of the same perturbers, or arrays of different perturbers in parallel, series, or both to achieve a specific result, for example, separation or concentration of particles into discrete streams or collection areas, sequential separation or concentration through multiple stages, and the like.

Thus, the methods and devices of the present invention employ at least one field non-uniformity within a channel resulting from at least one perturber, such as a projection, an obstacle, a patch, or a combination thereof. In some embodiments, the present employs at least one coherent array, which is a plurality of field non-uniformities that amplify the effect of a secondary flow field by judicious, substantially periodic repetition and may be used to prevent or decrease fouling, extend the operating envelope, and enhance of separations.

The present invention competes a primary flow field with a secondary flow field produced by the interaction of a particle with a field non-uniformity to separate particles. Unlike prior art methods which retain particles based on surface phenomena such as affinity, hydrophobic, affinity, and steric interactions, the methods and devices of the present invention separates (retains) particles as a result of interactions with field non-uniformities that occur throughout the three-dimensional space of the channel, not merely the regions in close proximity to surfaces of the channel. These field non-uniformities are the result of at least one perturber in a microchannel.

The dimensions of a field non-uniformity and therefore its associated perturber are based on the average size of the particle or particles of interest. To avoid surface interactions, the minimum channel dimensions, including regions surrounding obstacles, if present, is about several particle diameters, i.e., greater than about 3 diameters, preferably greater than about 10 diameters. Characteristics of a projection should be designed and the dimension of an obstacle or patch constrained to about equal to and preferably greater than about 3 times, the width of the flow area around the obstacle or patch so that the field extrema produced locally by the perturber vary by less than (preferably) 5% to 500% throughout the adjacent flow. A preferred size range of perturbers is about 10 to about 100 particle diameters. The obstacles or patches can be one-thousand or more times larger than the particles of interest, thereby facilitating the use of conventional photolithography to sort macromolecular particles. However, large ratios of the patch or obstacle sizes to particle sizes produce slower separations than those where the size ratios are closer to the more moderate preferred range, e.g., about 10 to about 100. For secondary flow mechanisms that derive their energy from the primary flow, e.g., dielectrophoresis vs. electrokinesis, large size ratios require relatively larger primary flow fields to be applied to drive the secondary flows than for more moderate size ratios. The width of the channel may be selected to achieve a desired throughput using methods known in the art. These ranges of size scales are offered only for practical guidelines and do not represent absolute physical limitations. The dimensions of the non-uniformities depend on the fabrication technique, particle size, and many other application-specific constraints which may be readily determined by one skilled in the art.

Depending on the type of field non-uniformity produced by the perturber, a particle can interact via a variety of mechanisms, including, for example:
a. Electrostatic: A particle experiences attractive or repulsive electrostatic forces with regions having a net opposite and like electric charge, e.g., in regions where electric Debye layers overlap, as in the phenomenon of Donnan exclusion, by which charged ions are repelled from regions of overlapped Debye layers.
b. Dielectrophoretic: A particle that is more or less conductive than its immersion fluid will respectively experiences an attractive force toward or repulsive force from regions of high electric field intensity. The resulting motion is dielectrophoresis.
c. Magnetophoretic: Particles having a magnetic permeability different from the immersion liquid will be influenced by non-uniform magnetic fields resulting in magnetophoresis.
d. Electrostrictive: Physically indistinct from dielectrophoresis, electrostriction is the motion of a polarizable object toward regions of high electromagnetic field intensity. Electrostriction at optical frequencies is one mechanism responsible for optical trapping.
e. Photophoretic: The absorption of electromagnetic energy can directly produce transport directly and can create a secondary flow indirectly from a number of mechanisms, e.g., from a perturbation in electrokinetic primary flow via manipulation of the electrical properties of the particle, and by changing particle mobilities by absorption-related heating and polarization induced alignment, orientation, and rotation.
f. Thermophoretic: a spatially non-uniform temperature field drives thermophoretic transport.
g. Entropic: Extensional and shearing flows do work by stretching elastomeric molecules, such as DNA. The resistance to this stretching produces an entropy-related secondary flow. This transport mechanism has been used in different implementations in “entropic trapping” devices. See Han & Craighead (2000) Science 288:1026-1029, which is incorporated herein by reference.
h. Mechanical: Similar to entropic mechanisms, shear, extension, and polarization in non-uniform flow and electric fields distort the structure of particles producing a restoring force related to bond (elastic) energy, not simply entropy. For molecules, the relaxation rate and spring constant of these restoring forces tend to be much higher than entropic effects. The amount of mechanical deformation and its relaxation rate may be a good indicator for the state of health of a variety of cells.
i. Inertial: DC driving: sedimentation: AC driving: acoustic streaming or acoustic focusing. Particles having a different density than their immersion medium will accelerate differently in non-uniform flow fields than the immersion medium. Centripetal accelerations produce primary-flow-transverse secondary flows. Flow-wise accelerations produce primary-flow-longitudinal secondary flows.
j. Nonlinear hydrodynamic: Particles in non-uniform flow fields generally experience explicit hydrodynamic effects from viscosity. Rotation and other kinds of primary flow can produce explicit hydrodynamic-related secondary flow of immersed particles.
k. Chemical: Chemical reactions and chemical conformational changes can affect transport when chemical potentials are spatially non-uniform. These non-uniformities include, for example, pH changes within finite Debye layers, photolytically generated reactant concentration variations, and optically driven excited-state population variations, and the like.

The types and details of the field non-uniformity produced by a perturber depend on the type of perturber, the nature of the applied field, and whether the perturber directly produces a field or produces non-uniformities in an otherwise applied field.

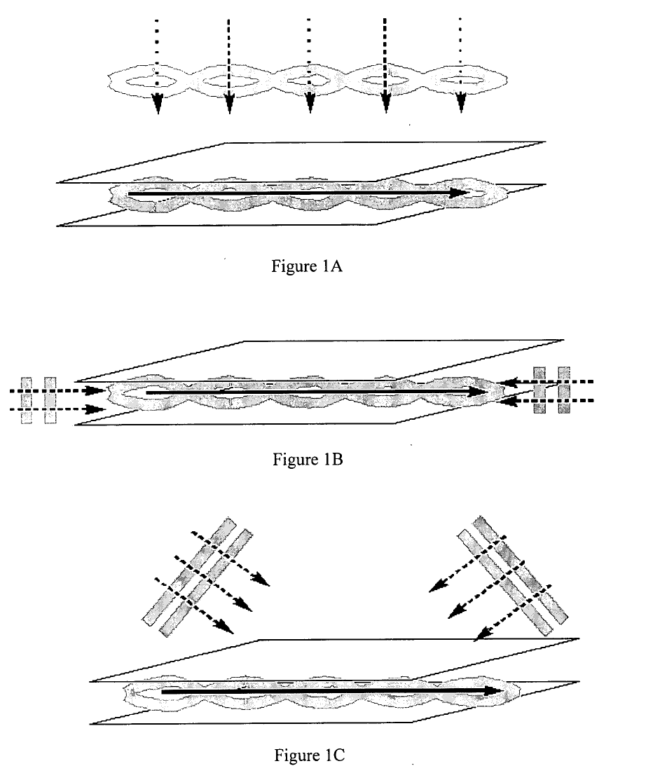

A projection is a perturber that is a non-uniform field projected externally into a channel, for example a non-uniform illumination pattern produced by an external diffractive optic or optical interference within the channel, a non-uniform acoustic excitation produced by an external non-uniform source or by acoustic interference within the channel. FIGS. 1A through 1C show schematic diagrams of how projections produce field non-uniformities in channels. The solid arrow indicates the primary flow. FIG. 1A shows a projection in which aspatially non-uniform field is prepared outside the channel and is projected into the channel to produce field non-uniformities. Examples include a magnetic field around a periodically poled magnet, or an optical field behind an external shadow mask. FIG. 1B is an example in which field non-uniformities are formed within the channel. Examples include interference, standing, or traveling waves such as a standing wave produced by an optical or acoustic interference pattern formed by a beam and one or more reflections or a traveling-wave interference pattern formed by an optical or acoustic wave and one or more frequency-shifted interfering wave. The interfering waves do not need to counter propagate, but merely must have different propagation vectors. FIG. 1C shows static or evolving interference patterns in a physical field projected into a channel by propagating two or more coherent acoustic or optical waves into a channel, e.g., by a variety of well known techniques such as splitting a laser beam and crossing the split beams in the channel, projecting a laser beam through a fixed holographic or diffractive element, or projecting a laser beam through an acousto-optic modulator, Bragg cell, or spatial light modulator of the optical phase, amplitude, or both into the channel. In each case, the result of a projection is a spatially non-uniform field in the flow channel.

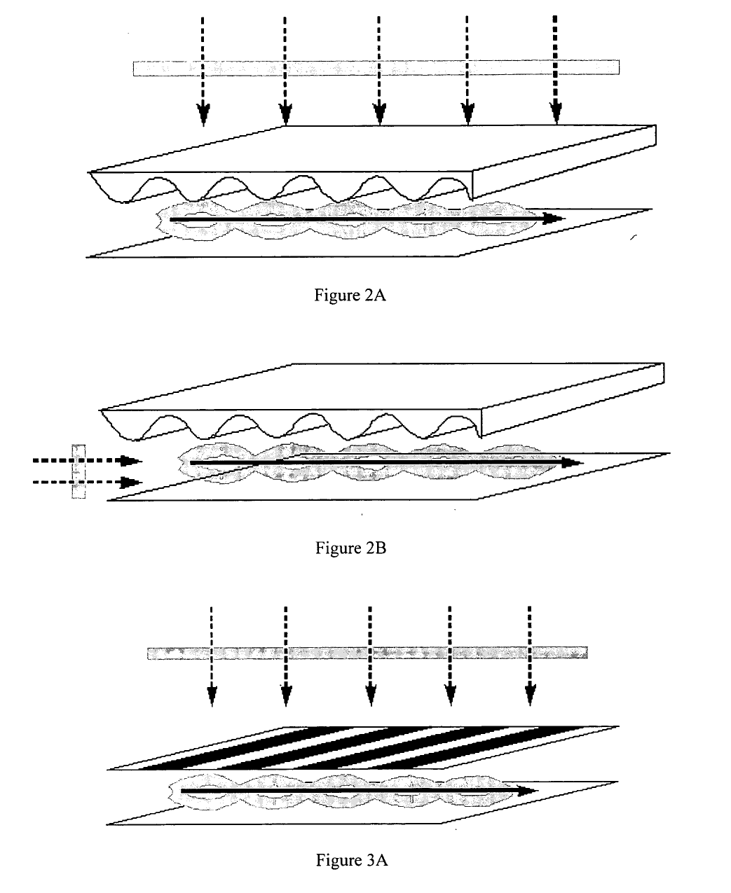

Obstacles are perturbers that are geometrical protrusions or cavities which generally produce field non-uniformities in externally applied fields, often fields that propagate through the fluid within the channels. FIGS. 2A and 2B show schematic diagrams of how obstacles modulate applied fields. The solid arrow indicates the primary flow. As used herein, the term “lensing” refers to any process by which a geometrical non-uniformity produces a spatial non-uniformity in a field including refraction, distortion, diffraction, concentration. FIG. 2A shows how obstacles can directly perturb an externally applied field that has a component normal to the flow channel, e.g., via lensing of an optical field, lensing of a magnetic field via magnetically permeable obstacles, lensing of transverse electric field via electrically conductive obstacles, lensing of a high-frequency electric field by insulating, conductive, or insulated conductive obstacles. FIG. 2B shows how obstacles can also directly perturb an axial applied field or field that propagates through or is associated with the fluid within the channel, e.g., concentration and rarefaction of an axial electric field via insulating obstacles, concentration of a hydrodynamic velocity field via impermeable obstacles.

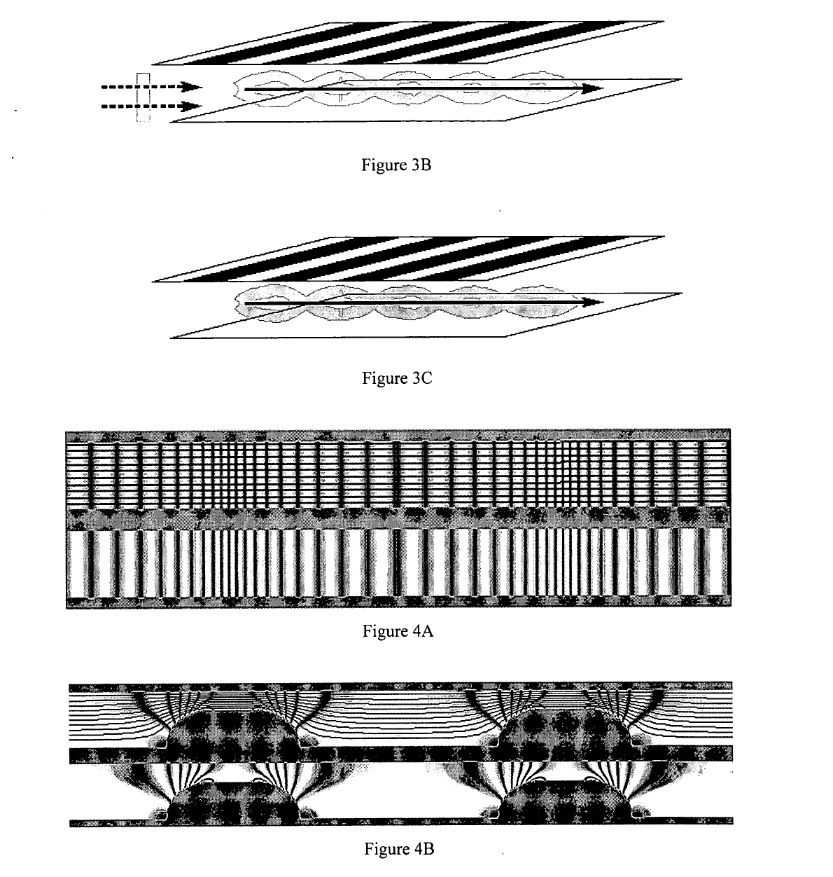

Patches are perturbers that regions that are substantially flush with channel surfaces which produce field non-uniformities in externally applied fields or directly apply a field non-uniformity, e.g., via direct application of electric, optical, acoustic, or thermal perturbations. FIGS. 3A through 3C show schematic diagrams of how patches modulate applied fields and directly drive non-uniform fields. The solid arrow indicates the primary flow. FIG. 3A shows how patches can perturb the propagation of an external applied field into the flow channel, e.g., via shadow masking of a propagating or evanescent optical field by periodically absorbing, reflecting, or patches; modulating a magnetic field by magnetically permeable patches, and the like. As used herein, a “fluid-associated field” is a field that is linked to the fluid, e.g., the flow velocity field, the chemical concentration field, the fluid density field, and the like. FIG. 3B shows how patches can perturb the propagation of an axial or fluid-associated field, e.g., via modulating an electric field with conductive or insulating patches, and the like. FIG. 3C shows how patches can directly drive a spatially non-uniform field, e.g., by applying fields directly or can perturb intrinsic or spontaneously forming fields within a channel, e.g., via spatially modulating the surface charge density or sign.

Table 1 lists examples of perturber mechanisms that can be used to producing various secondary flows according to the methods and devices described herein:

TABLE 1
Examples of perturbation techniques for producing various secondary flows

| Secondary flow: | Projection | Obstacle | Patch |
|---|---|---|---|
| Nonuniform field | techniques | techniques | techniques |
| Electrophoresis: | Photoionization by non- | Obstacles produce | Non-uniform zeta |
| Charge density | uniform applied optical | variable Debye-layer | potential, permeability, |
| | field | overlap | conductivity, |
| | | | illuminated photo- |
| | | | sensitive surface, |
| | | | insulated gate |
| | | | electrodes modulate |
| | | | charge density of fluid |
| | | | or particle |
| Dielectrophoresis: | (see electrostriction) | Insulating or | Conductive patches |
| Electric | | conductive obstacles | directly apply or distort |
| | | distort electric field | externally applied |
| | | applied through fluid | electric field |
| Magnetophoresis: | Produced by | High-permeability | High-permeability |
| Magnetic | periodically poled | obstacles distort | patches modulate |
| | permanent magnets or | applied magnetic field; | externally applied |
| | electromagnets, non- | low-permeability | magnetic field strength. |
| | uniformly shielded | obstacles perturb field | Magnetic patches |
| | magnet or magnets | in ferrofluid | directly produce field. |
| Electrostriction: | Externally applied non- | Refractive or reflective | Shadow masking, |
| Optical, electric | uniform illumination, | lensing or waveguiding | diffraction, leaky |
| | e.g., interference | of an applied | waveguiding of applied |
| | fringes | illumination | illumination |
| Photophoresis: | (see electrostriction) | (see electrostriction) | (see electrostriction) |
| Optical | | | |
| Thermophoresis: | Externally applied non- | Joule heating of | Conductive or |
| Temperature | uniform thermal field, | immersion fluid in non- | insulating patches |
| | absorption of a non- | uniform channel; | modulate Joule heating |
| | uniform illumination | refractive or reflective | from patch-applied or |
| | field, applied non- | lensing of applied | fluid-conducted electric |
| | uniform infrared | illumination with | field; resistance |
| | heating. | absorption | heating of conductive |
| | | | patches; illumination |
| | | | shadow-masking or |
| | | | diffraction with |
| | | | absorption |
| Entropic trapping: | Projected temperature | Obstacles directly | Patches apply |
| Velocity | gradients produce | perturb flow | temperature variations |
| | entropic forces on | streamlines | that produce entropic |
| | particles | | forces on particles |
| Mechanical distortion: | Particle conformation | (see entropic trapping) | Patches apply or |
| Velocity | or geometry is | | modulate fields that |
| | perturbed thermally or | | geometrically distort |
| | via optical excitation | | particles |
| Nonlinear | Projected fields | (see entropic trapping) | Patches apply or |
| hydrodynamics: | produce particle | | modulate fields that |
| Velocity | alignment or orientation | | produce alignment or |
| | | | orientation |
| Sedimentation | Projected fields change | (see entropic trapping) | Patches induce fields |
| (DC inertia): | drag coefficient via | | that change drag |
| Velocity | particle alignment or | | coefficient via |
| | orientation | | alignment or orientation |
| Acoustic streaming (AC | Externally applied | Obstacles having | Acoustic field actuated |
| inertia): | spatially non-uniform | different acoustic | by actively driven |
| Acoustic, velocity | acoustic field; applied | impedance than fluid | patches; patterned |
| fluctuations | acoustic field with | perturb acoustic field | non-uniform acoustic |
| | standing waves | | impedance modulates |
| | | | directly applied or fluid- |
| | | | propagated acoustic |
| | | | field |
| Chemical modification: | Optically driven | Obstacles produce | Patterned surface |
| Chemical potential, | chemical potential, | strains that modulate | chemistry; patterned |
| concentration | excited or activated- | reaction rates | photoactivated surface |
| | state population | | with uniform |
| | modulation | | illumination |

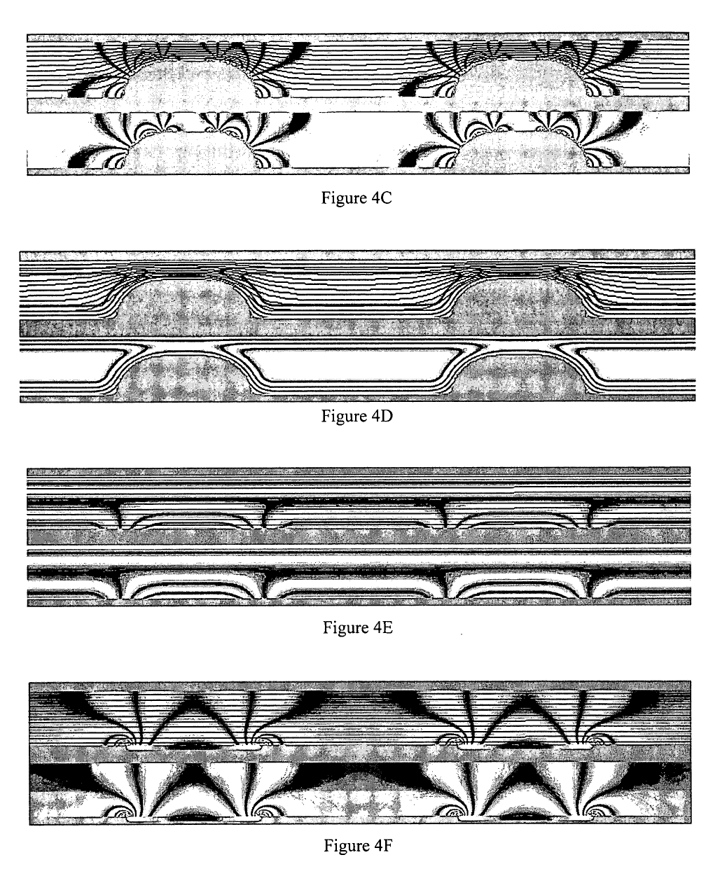

For illustration, the interactions of a particle with field non-uniformities in a channel can be expressed via a particle perturbation potential energy field which is the surplus or deficit potential energy that a particle possesses at as a result of the interactions. The gradient of these potentials is the secondary flow force and the resulting component of particle motion produced by these forces is the secondary flow. FIGS. 4A through 4F show cross-sections of particle potential energy perturbations produced by examples of perturbers according to the present invention. The fringes are the contours of constant potential energy and the overlaid lines represent unperturbed streamlines. The perturbation force and terminal velocity associated with these potentials are normal to the fringes. Particles gain or lose kinetic energy as they cross fringes and are redirected from their unperturbed streamlines as they cross fringes obliquely. The cumulative effects of these perturbations or interactions are secondary flows that retain and redirect particles from the primary flow by an amount depending on the physical mechanisms for the interaction and the relevant particle and immersion-fluid properties. The dependence of the secondary-flow magnitude on particle properties enables sorting of the particles by these properties.

For simplicity, the figures show obstacles or patches only at the bottom surface of a microchannel. Nevertheless, obstacles or patches on more than one microchannel wall, e.g., both the top and bottom surfaces with aligned and out-of-phase patterns, are contemplated herein. Similarly, alternative perturber geometries are contemplated herein, e.g., patterned channel-spanning posts, patterned valleys, and different obstacle shapes, are substantially periodic at least over domains within a device.

FIG. 4A shows the potential energy distribution from projection of a field having a sinusoidal variation experienced by particles that are attracted or repelled by the field in proportion to the gradient of the square of the field, e.g., particle electrostriction produced by an optical interference pattern projected into the channel or the field behind periodic patches that alternately block and pass an applied illumination, or “shadow mask”. Particles having an index of refraction greater and less than that of the immersion liquid are respectively attracted to and repelled from regions of high field intensity. If the primary flow field is electrokinetic, this interaction benefits from having minimal hydrodynamic dispersion and minimal variation in the magnitude of the secondary effect as a result of the negligible variation in illumination intensity with channel depth.

FIG. 4B shows the electrostatic potential energy resulting from dielectrophoretic forces produced by insulating, impermeable ridge obstacles in a fluid having an applied electric field, which is also similar to centrifugal potential energy contours over obstacles in an applied hydrodynamic flow field. Specifically, FIG. 4B shows the electrostatic perturbation field associated with dielectrophoresis produced by applying an electric field through a conductive fluid in an insulating microchannel. The ridges in the channel concentrate and rarefy the applied electric field. The dielectrophoretic potential energy perturbation is proportional the electric field intensity. Particles that are insulating relative to the fluid experience a high potential energy above a ridge. These particles are retained slightly (lag the primary flow) at the leading edge of each ridge. Particles that are relatively conductive experience a low potential energy above a ridge. These particles are retained slightly at the trailing edge of each ridge. A somewhat similar perturbation potential energy distribution is produced when particles that are negatively or positively buoyant are forced past substantially impermeable, but not necessarily insulating ridges at speeds where their inertia becomes important. In such a system positively and negatively buoyant particles are respectively retained at the leading and trailing edges of the ridge.

FIG. 4C shows entropic potential energy resulting from flow-induced particle distortion produced by substantially impermeable ridge obstacles. Specifically, FIG. 4C shows the entropic or mechanical potential energy field experienced by a deformable particle undergoing flow-induced distortions. The extensional flow near the stagnant region in front and back of a particle and the regions where streamlines converge and diverge at the upper sides of the ridges are regions of high potential energy. The distortion relaxation of the particle is assumed to be rapid as compared to the passage time. A potential energy field more similar to FIG. 4B is experienced by a particle whose distortion relaxation is slow compared to its passage time. The difference between these potential energy fields allows particles to be sorted on the basis of their recovery time to a mechanical distortion as well as the magnitude of their mechanical distortion.

FIG. 4D shows electrostatic potential energy resulting from periodic electric double layer overlap over ridge obstacles having a substantially uniform surface charge density. The fringes along the channel boundaries show the electric double layer thickness which is generally about ¼ of the deep channel depth. Specifically, FIG. 4D shows the electrostatic perturbation potential of a charged particle passing through a channel in which the net-charged electric double layers (Debye layers) overlap in the region above the ridges. Particles having the same sign of charge as the boundary experience a repulsive force that retains them at the leading edge. If this force is stronger than the primary mobilization field, such particles are trapped upstream of the ridge in an effect commonly known as Donnan exclusion.

FIG. 4E shows the electrostatic potential energy in a channel having nearly overlapped electric double layers resulting from patch perturbers having periodically pooled charge densities at the bottom surface. Specifically, FIG. 4E shows the electrostatic perturbation potential of a charged particle passing through a channel in which the surface charge density of the lower surface is periodically pooled, e.g., by patterning a coating of a oppositely charged molecules on a surface, by patterning a coating that enhances or blocks a fraction of the surface charge, by modifying the native surface of a channel through standard techniques such as e-beam, plasma, ion, and photon treatments, by constructing channels of block co-polymer having different pKAs, by dynamic surface coatings including surfactants, epoxilation, sulfonation, silanation, amination, and the like. In the figure, the upper surface of the channel is not modified, so the perturbation occurs only at the bottom surface. Particles will generally be retained when forced by the primary flow toward a region having a like surface charge.

FIG. 4F shows electrostatic potential energy in a channel resulting from patch perturbers having periodically patterned conductivity at the bottom surface. Specifically, FIG. 4F shows the perturbation potential energy of a charged particle in an otherwise insulating channel having patterned surface conductivity (the dips in the lower surface are filled with relatively conductive material) under the influence of an electric field applied through the fluid. The conductive regions could, for example, be electrodes patterned by any of a number of well-known techniques. FIG. 4G shows potential energy resulting from conductive ridges. Specifically, FIG. 4G shows an alterative geometry to that in 4F which contains obstacles covered or constructed from conductive patches. The geometry of the ridge significantly smoothes the electrostatic potential distribution, reducing the dispersion associated with the retention mechanism at the cost of additional hydrodynamic dispersion from the primary flow resulting from the presence of the obstacle. FIG. 4G also shows an example of an obstacle that has at least one different property than the surrounding surface of the microchannel (similar to conductive patches).

Highly elongated projections (fringes), obstacles (ridges and valleys), and patches (stripes) are preferred perturbers for many CNC designs because they facilitate the construction of channels having useful and relatively easily specified distributions of field non-uniformities. An elongated perturber can be characterized by a local axial angle that is locally aligned with the most narrowly spaced sides of the perturber and a local normal direction which is at right angles with the local axial direction. Such elongated perturbers typically have a length that is greater than three times the width and have a maximum radius of curvature greater than three widths throughout the majority of the perturber. The dimensions and shapes of the perturbers may be modified, particularly in regions where particles are to be collected or released and at the ends of the perturbers. There is no maximum aspect ratio except that needed to facilitate fabrication, mechanical strength, and dimensional accuracy. A practical upper bound of aspect ratio is limited by sag and deflection of unsupported boundaries of the channel containing the perturber. For unpressurized channels, the maximum ratio of channel width to channel depth with simple etching has been found to be about 10^{3,} while about 10² or lower are preferred. Highly pressurized channels require lower ratios of unsupported channel width to height. These ratios can be increased by the addition of internal supports, the use of thick substrates, or the use of external stiffeners.

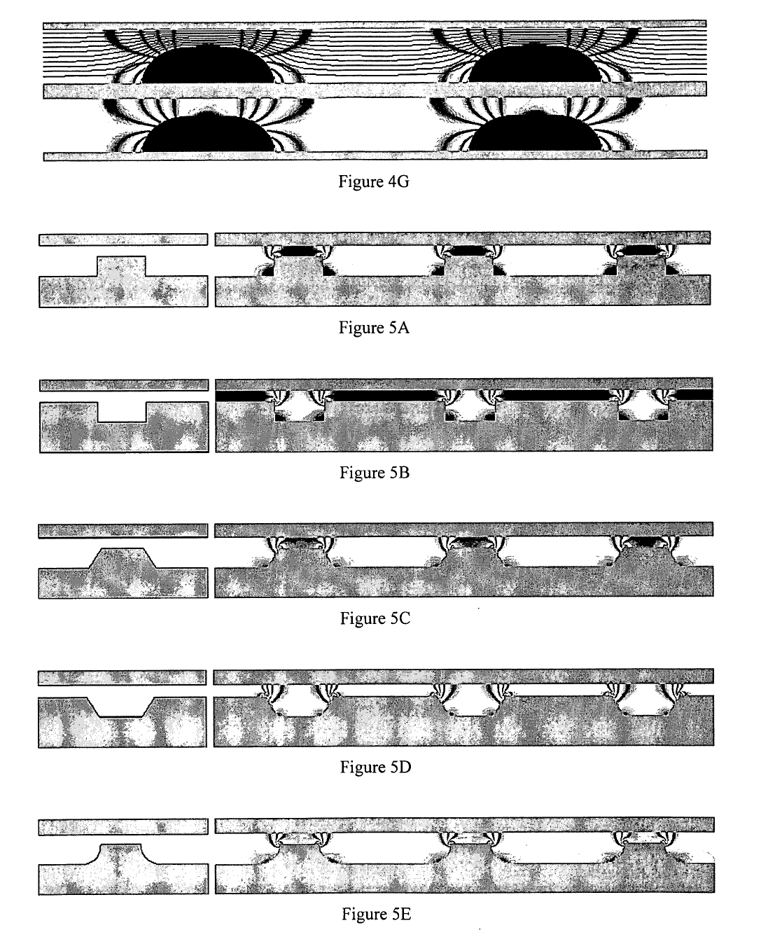

To clarify the action of obstacle perturbers, which generally modulate an otherwise applied field, FIGS. 5A through 5H show examples of the cross-sections of insulating obstacles according to the present invention and their effect on the dielectrophoretic potential energy of particles flowing past the obstacles via the concentration effect of the obstacles on an electric field applied through the channel fluid. The cross-sectional obstacle shapes, typically determined by the means of their fabrication, affect their behavior and performance. The right panel of each figure shows the cross-sections of the particle potential energy perturbations produced by non-uniform fields. The fringes are contours of constant potential energy. FIG. 5A generally represents a cross-section of an obstacle formed using a high aspect ratio etching procedure, such as reactive ion etching, thick photoresist etching (e.g., SU-8), LIGA, conventional machining and replication technologies such as casting, molding, stamping, embossing, and injection molding, and others known in the art. FIG. 5C generally represents a cross-section of an obstacle formed using an anisotropic low-aspect ratio fabrication technique such as wet etching in a crystalline substrate, reactive ion etching, and others known in the art. FIG. 5E generally represents a cross-section of a obstacle formed using isotropic etching techniques such as wet etching, and others known in the art. FIG. 5G generally represents a cross-section of an obstacle formed using molding or stamping methods known in the art. These shapes and others may be readily formed using methods known in the art.

Generally, obstacles with sharper edges, such as those shown in FIG. 5A, produce stronger and more localized field non-uniformities, which can adversely affect performance by creating significant differences in the magnitude of particle interactions per obstacle versus channel depth and an increased tendency of particle adhesion. Obstacles with rounder edges, such as those shown in FIG. 5G, produce weaker, less localized field non-uniformities that generally reduce the variations in the magnitude of particle interactions with depth. The particular particle and fluid to be manipulated, and practical considerations such as the tolerable electric field strength, voltage, pressure, shear, and the like, influence the choice of obstacle shape. If careful tailoring of the obstacle cross-section is needed for a particular application, post or column shaped obstacles may be preferable to ridge or valley obstacles, since their cross-section can be defined photolithographically, rather than dictated by the choice or etching or deposition technique. Those skilled in the art may readily select a particular shape in order to obtain a desired type and degree of particle separation and analysis in accordance with the invention herein.

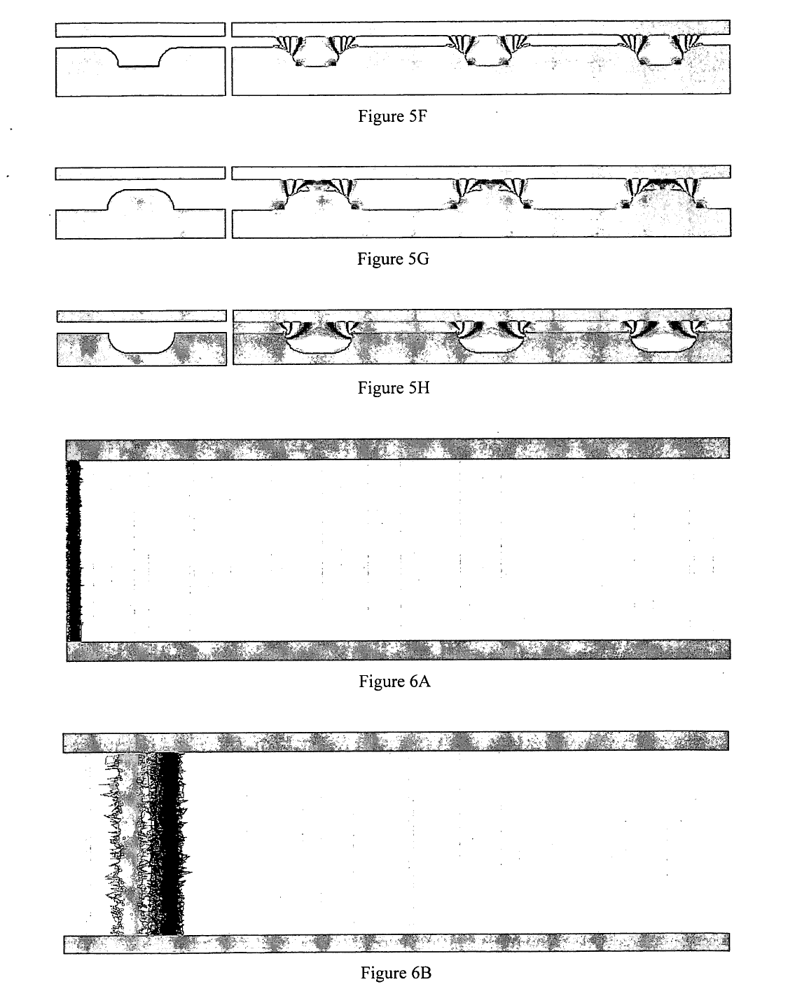

FIGS. 6A through 6E represent a simulation of a separation using a coherent array according to the present invention. In particular, a “time-of-flight” separation is exemplified. As used herein, “time-of-flight” refers to separations, wherein the perturbers span the width of the microchannel at an angle that is substantially perpendicular to the primary mobilization field, thereby resulting in separations of similar particles in bands that are substantially parallel to the coherent array. In the figures, fluid flow is from left to right. FIG. 6A is at time 0 wherein a fluid comprising three different particles (differentially interact with the field non-uniformities created by the ridges) are introduced to the coherent array. FIGS. 6B through 6E are consecutive periods of time. As shown in FIG. 6B, the particles which strongly interact with the field non-uniformities, i.e., experience 2× the potential field perturbations experienced by reference particles), as represented by the light gray band, begin to lag behind and separate from the other particles. As shown in FIG. 6C, the particles which strongly interact are completely separated from the other particles and the reference particles which weakly interact with the field non-uniformities, i.e., experience 1× the potential field perturbations, as represented by the dark gray band, begin to lag behind and separate from the particles that do not interact with the field non-uniformities, as represented by the black band. FIGS. 6D and 6E show further separations of the three particle types. Time-of-flight separations may be performed using a coherent array of perturbers of any type (projections, obstacles, and patches). In preferred time-of-flight embodiments, the secondary flow is substantially aligned with the primary flow so that the main effect of the secondary flow is to retard the motion of particles down the channel. In the case of projected straight fringes, ridge-like obstacles, or linear patches, this alignment of the secondary flow can be achieved by orienting the long axis of the perturbations normal to the primary flow, as indicated by the grayscale background in FIG. 6.

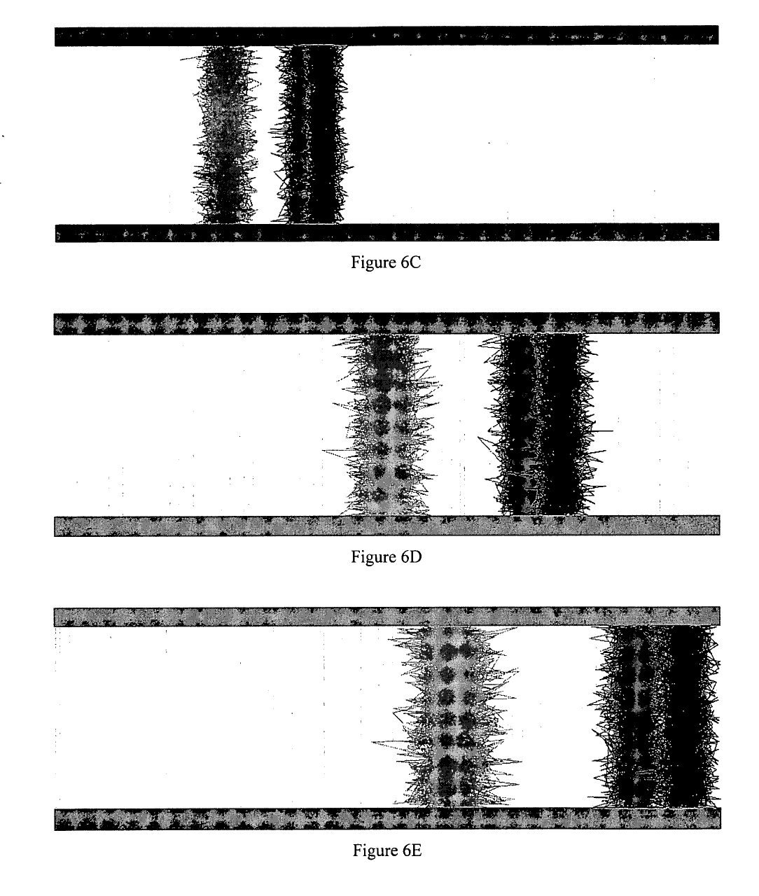

In alternative embodiments, the coherent arrays may be placed so that the perturbers and consequently the secondary flows have a substantial component transverse to the primary flow, as shown in FIGS. 7A and 7B, which may be used for concentration or “spectrometric” separations, wherein particles are separated transversely by how much they interact with the field non-uniformities. In FIGS. 7A and 7B the primary flow is from left to right. FIG. 7A shows the tracks of particles that weakly interact with the competing mobilization field. The weak secondary flow results in particle migration transverse to the primary flow. The stronger it interacts with the non-uniform-field, the more closely a particle follows the axis of the associated perturber. These interaction produce greater angular deflections from the primary flow up to the limit at which the interaction is so strong that particles are inhibited from crossing the field non-uniformities and propagate substantially along the axis of the associated perturber, as shown in FIG. 7B.

Spectrometric separations may be performed using a coherent array which spans the width of the fluid of interest at an angle. In preferred spectrometric embodiments, the fluid flow is at an angle with respect to fringe-like projections, ridge-like obstacles, or stripe-like patches. In other preferred embodiments, the fluid flow is at a small (about <15 degree) angle with respect to a coherent array produced by a plurality of perturbers such as posts, holes, protrusions, cavities or patches such that the secondary flow of particles causes the particles to be deflected from the toward the plurality of perturbers.

A particle is impeded from crossing such a perturber when the largest local normal component of the secondary flow force successfully opposes the local normal component of the primary flow force. As used herein, this condition is called “inhibition”. As used herein, the “inhibition threshold” is the locus of conditions that separate the case where a given particle does and does not experience inhibition.

The local angle of the long axis of a perturber with the primary flow in general affects the particle transport past the perturber and can affect the magnitude of the secondary flow force produced by the perturber. The nature of these angle effects depends on the how the normal force produced by the perturber, δ, scales with the primary mobilization field magnitude, μ. The perturbation scaling can be expressed approximately as a power of μ, i.e., δ˜μⁿ. The dependence of the ratio of the secondary flow force to primary flow force across a ridge, γ, with the local angle, θ, between the perturber axis and primary flow direction is generally γ˜sinⁿ⁻¹ θ.

In the simplest case, the perturbation forces normal to the perturber axis are substantially independent of the primary flow magnitude, so the exponent of the power law, n, is zero, e.g., optically induced electrostriction and photophoresis vs. hydrodynamic or electrokinetic primary flow, dielectrophoretic forcing vs. hydrodynamic primary flow, and the like. In this case, there is no angle dependence of the secondary flow normal force, but there is a sinusoidal dependence of the force applied by the primary flow across the perturber on the local axial angle, so the ratio of the secondary flow normal force to primary flow normal force scales like the cosecant of the local axial angle. Thus the inhibition effect of a ridge is reduced by angling the ridge axis toward a right angle to the primary flow.

In some situations, the primary flow couples weakly to the secondary flow force, e.g., via convective charge polarization effects within Debye layers, particle alignment or distortion effects, and the like. Provided the actual exponent n<1, the angle effects of the perturber will remain qualitatively as described for n=0, but the variation of the inhibition effect with angle reduces until as n→1, there is no angle dependence of inhibition.

The power n>1 implies that the primary flow contributes nonlinearly to the secondary flow, i.e., the secondary flow is partly or completely powered by the primary flow. In this range the angle dependence switches such that the competition between primary and secondary flow becomes more favorable for the secondary flow as the perturber axis becomes more misaligned with the primary flow, reaching a peak when oriented normal to the primary flow. This angular variation is qualitatively the opposite of that for n<1.

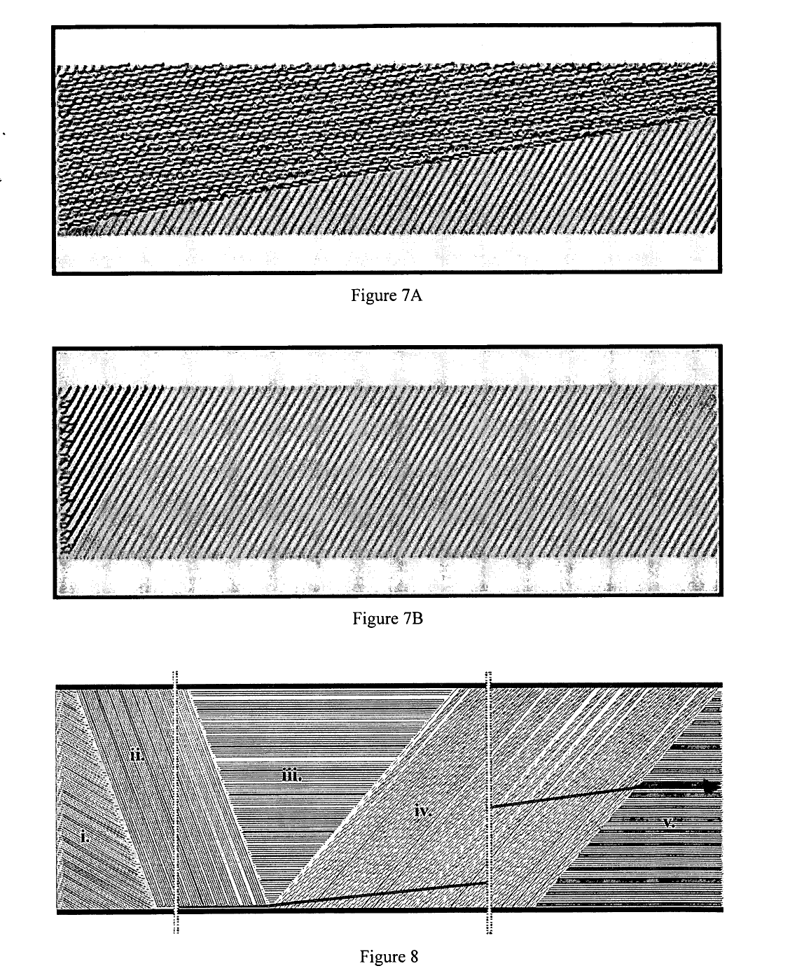

FIG. 8 shows a CNC particle sorter or spectrometric device embodiment showing examples of various domains of arrayed perturbers and uses thereof. As provided herein, “a domain” is a plurality of perturbers in an array. The primary flow is from left to right. This particular design applies for secondary flows like those in FIG. 7A that redirect, but do not inhibit particles and that scale greater than the first power of the primary flow, i.e., n>1. Completely analogous devices can be constructed for primary and secondary flows in which n<1 by tilting the perturbers in domain ii so they are more aligned with the primary flow than those in domain iv. A dilute, initially uniform concentration of particles is concentrated along the lower wall of the sorter by the perturbers in ii. The domain ii can be eliminated if particles can be introduced through a secondary side port at this wall. This stripe of concentrated particles then propagates at an angle through domain iv that depends on particle properties and emerges in domain v at a particle-property-specific transverse location. The optional domain i only weakly deflects particles and is used mainly for impedance matching purposes. Domain iii has no transport effect other than to match the channel impedance between domains ii and iv. Domain v also matches impedance and can optionally comprise partitions that isolate the various particle fractions emerging from iv.

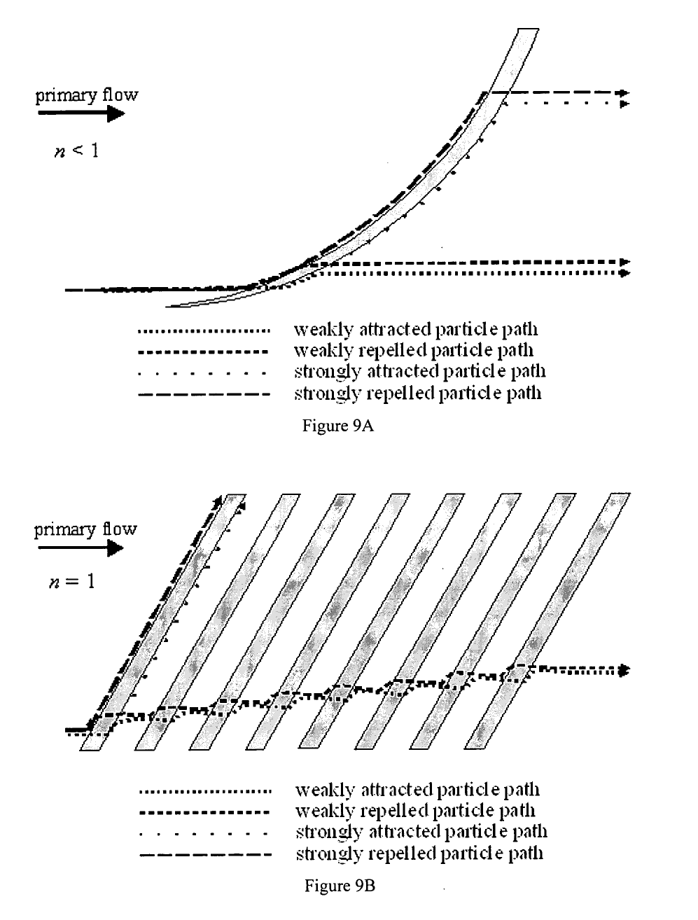

The angle dependence of the inhibition thresholds enables a different class of particle spectrometers. FIG. 9A shows an example of a CNC spectrometer according to the present invention that disperses particles in the direction transverse to the primary flow such that particles flow in a localized stream to a region where the perturber is substantially aligned with the flow and gradually curves in the direction normal to the flow. Inhibited particles propagate along the perturber until the local axial angle with the primary flow is sufficiently high, at which point such particles pass the perturber and resume substantially following the primary flow at a transverse position that correlates to the amount of interaction with the perturber. It is possible for particles to be inhibited at all axial angles for a given primary flow and perturber, requiring some adjustment to the other parameters, e.g., a primary flow increase or direction change or a reduction in the perturbation, e.g., by reducing a projected non-uniform field in order to elute the particles. Theoretically, all particles can be inhibited by such a ridge provided the axis is sufficiently close to alignment with the primary flow. However, practical fabrication tolerances and device aspect ratios typically limit the range of useful axial angles to greater than about 1° to about 5°.

The limit n=1 implies a first-order dependence of the secondary flow on the primary flow. Such dependence can be introduced if the perturbation interaction couples to the primary flow, for example if the secondary flow is produced by optically perturbing the surface charge density of a particle in an electrokinetic primary flow. Because the normal components of the secondary and primary flows have the same angle dependence, the inhibition threshold has no angle dependence. A spectrometer made from such a perturber, cannot exploit an angle dependence of the inhibition threshold. Instead, the perturber must either operate as a disperser below the inhibition threshold, as shown in FIG. 9B or modulate the primary or secondary flow field in time, or a combination of these actions.

Theoretically, several important phenomena obey the scaling n=2, e.g., a dielectrophoretic secondary flow vs. an electrokinetic primary flow and an inertial secondary flow vs. a hydrodynamic primary flow, and the like. At this scaling, the primary force normal to the ridge scales sinusoidally with angle, but the secondary force scales as the sine squared, so the ratio of the secondary flow normal force to primary flow normal force scales like the sine of the local axial angle with respect to the primary flow. In this case, a canonical spectrometer can be constructed from a ridge that curves as shown in FIG. 9C. Particles initially encounter a perturber at a steep angle, at which the secondary flow is relatively strongest. Particles then propagate along the perturber until the perturber is sufficiently aligned with the flow that the secondary flow force is reduced below the primary flow force (from which the second-order, secondary flow force is derived) and the particles escape the perturber. A discussion of the design of ridge and valley obstacles for the special case of a dielectrophoretic secondary flow vs. an electrokinetic primary flow is contained in U.S. Patent Application Publication No. 20050072676, which is incorporated herein by reference. That design discussion applies quantitatively to secondary and primary flow systems obeying the scaling n=2 and qualitatively to systems obeying the scaling n>1 with the more general class of highly elongated perturbers described in this disclosure.

Table 2 shows the range of n, a measure of the coupling between the primary and secondary flows for various parings of these flows. The parameter n quantifies coupling between the primary and secondary flows, with n=0 indicating no coupling and n=2 indicating strong coupling.

TABLE 2
Ranges of n for various secondary and primary flows

| Secondary flow mechanism | Hydrodynamic primary flow | Electrokinetic primary flow |
|---|---|---|
| Electrophoresis | 0 ≦ n ≦ 1; n = 0 (ideal) | 0 ≦ n ≦ 1; n = 0 (ideal) |
| Dielectrophoresis | 0 ≦ n ≦ 1; n = 0 (ideal) | 1 ≦ n ≦ 2; n = 2 (ideal) |
| Magnetophoresis | 0 ≦ n ≦ 1; n = 0 (ideal) | 0 ≦ n ≦ 1; n = 0 (ideal) |
| Electrostriction | 0 ≦ n ≦ 1; n = 0 (ideal) | 0 ≦ n ≦ 1; n = 0 (ideal) |
| Photophoresis | 0 ≦ n ≦ 1 | 0 ≦ n ≦ 2 |
| Thermophoresis | 0 ≦ n ≦ 1 | 0 ≦ n ≦ 1 |
| Entropic trapping | 0 ≦ n ≦ 1 | 0 ≦ n ≦ 1 |
| Mechanical distortion | 1 ≦ n ≦ 2; n = 2 (ideal) | 1 ≦ n ≦ 2; n = 2 (ideal) |
| Sedimentation (DC) | 1 ≦ n ≦ 2; n = 2 (ideal) | 0 ≦ n ≦ 1; n = 0 (ideal) |
| Acoustic streaming (AC) | 0 ≦ n ≦ 2; n = 0 (ideal) | 0 ≦ n ≦ 1; n = 0 (ideal) |
| Nonlinear hydrodynamics | 1 ≦ n ≦ 2; n = 2 (ideal) | 0 ≦ n ≦ 2; n = 2 (ideal) |
| Chemical modification | 0 ≦ n ≦ 2 | 0 ≦ n ≦ 2 |

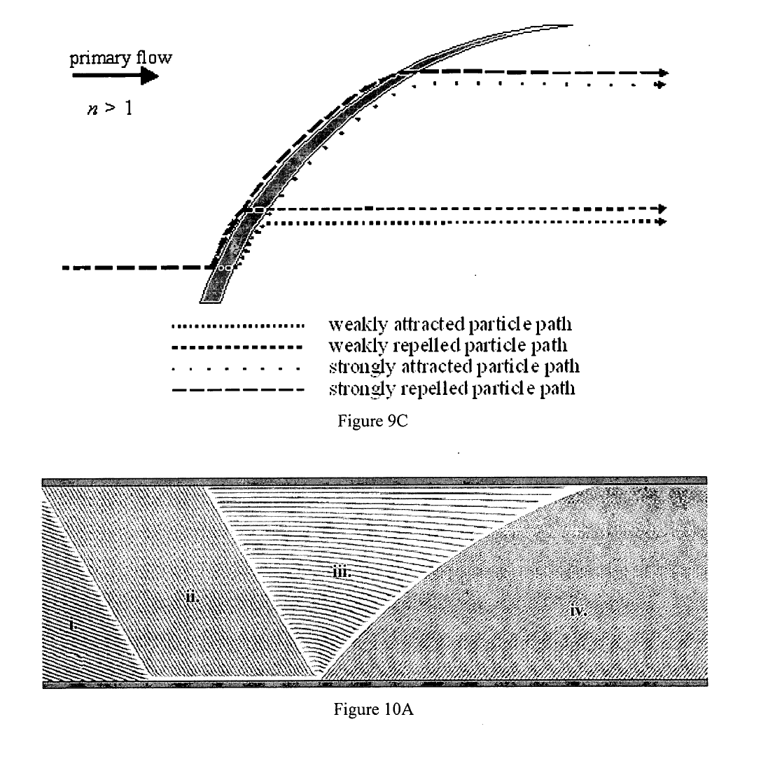

FIG. 10A is a diagram of a multiple-domain continuous-flow spectrometer according to the present invention in which n>1 and particles are substantially inhibited by their interactions with the non-uniform fields, depending on the local angle of the perturber with the primary flow. Particles and the primary flow are introduced at the left of the channel. Domain i is primarily intended for impedance matching and otherwise only weakly deflects particle trajectories downward. This domain is largely passive and optional. Domain ii deflects all particles of interest toward the side. This domain can be excluded if particles are introduced to the primary flow via a secondary inlet port at this side. Domain iii again is a largely passive and optional simply for impedance matching, Domain iv consists of an array of curved perturbers. The arraying provides for robustness against fouling and improved resolution, particularly for strongly diffusing particles. The curvature spatially disperses particles as described and shown in FIG. 9C. A corresponding system for an interaction in which n<1 would have the opposite curvature in domain iv, as shown in FIG. 9A and a decrease in the angle between the perturber axes and primary flow in domain ii, among other possible optional variations. Particles flowing out of the device at the right can be detected or collected into separate fractions using geometries such as bins and separate ports and the like, as is well known in the art. FIG. 10B shows a numerical transport simulation of a polydisperse fluid comprising diffusive particles in the device shown in FIG. 10A. The particles are spatially dispersed according to their degree of interaction with the non-uniform fields produced by the perturbers as indicated in FIG. 10B. Ranges of particles having weaker and stronger interactions are respectively highlighted in white and black.

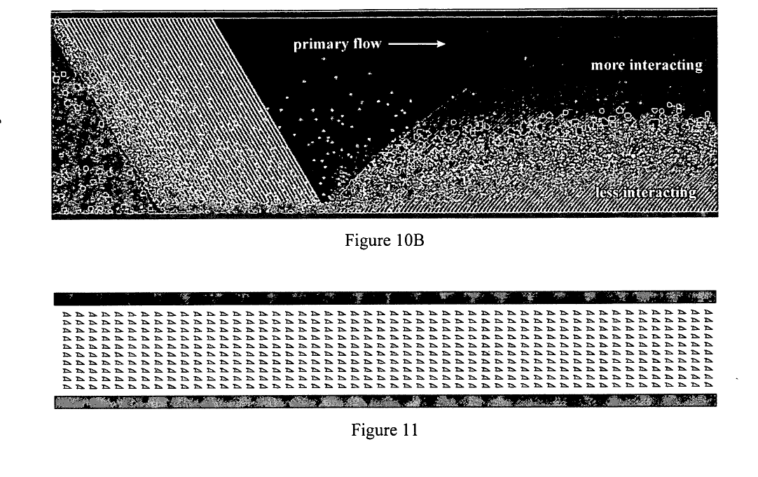

Because the region of particle interaction with a perturber depends on the properties of the particle, asymmetrical perturbers can have a variety of novel functions. FIG. 11 shows one example of an asymmetric perturber array. Because the perturbers are asymmetrical transverse to the primary flow, they will generally produce a net transverse secondary flow. For flow in a given direction, the details of the secondary flow depend on whether the interaction is attractive or repulsive, since the particles will dwell respectively at the downstream and upstream sides of the perturbation, where the non-uniformities and consequent secondary flow have different characteristics. Moreover, the secondary flows produced via the array of perturbers in FIG. 11 will transport a given particle differently if the primary flow is from left to right or from right to left as a result of these differences between the left and right sides of the perturbers. This difference allows such asymmetric arrays to be used as novel direction-sensitive transporters, ratchet devices, or devices for cyclically separating particles via applying a primary flow having a periodic reversal.

The devices of the present invention are readily fabricated using methods known in the art including techniques conventionally used for silicon-based integrated circuit fabrication, embossing, casting, injection molding, and the like. See e.g. Becker, et al. (1986) Microelectr. Engineer. 4:35-56, which is herein incorporated by reference. Other suitable fabrication techniques include photolithography, electron beam lithography, imprint lithography, reactive ion etching, wet etch, laser ablation, embossing, casting, injection molding, and the like. See e.g. Becker, et al. (1998) Microelectr. Engineer. 8:24-28, which is herein incorporated by reference.

The devices of the present invention may be fabricated from materials that are compatible with the conditions present in the particular application of interest. Such conditions and considerations include pH, temperature, application of organic solvents, ionic strength, pressure, application of electric fields, surface charge, sticking properties, surface treatment, surface functionalization, bio-compatibility, and the like. The materials of the devices may be chosen for their optical properties, mechanical properties, and for their inertness to compounds to be exposed thereto. Such materials include glass, fused silica, silicone rubber, silicon, ceramics, polymers, and the like.

In some embodiments, particles are unloaded from the array using methods known in the art, including microfluidic channels at the end of the array and are then be routed for further use. In particular, binning channels may be employed to divert a fluid stream of interest to a detection area for observation or visualization, to a reaction area or another device for further manipulations including chemical and physical reactions, and the like.

Embodiments diagrammed herein show only the active region of the CNC flow channel, not the means of introducing or collecting particles, primary flows, or means of providing for flushing, cleaning, elution, and the like. Thus, it should be noted that the devices of the present invention may further comprise multiple ports, offset-t structures, electrodes, and other microfluidic structures and means known in the art for implementing sample introduction, manipulation and analysis.

Moreover, embodiments diagrammed herein do not show the means of or external apparatus for applying fields and the primary flow. The design and possible integration of such external apparatus is obvious or known in the art and can be conveniently and reliably accommodated.

In preferred embodiments, portions of the devices of the present invention are optically transparent such that optical detection methods known in the art, including fluorescence detection and imaging using inverted-optic microscopes, may be used. In preferred embodiments, the fluid flows and separations are monitored in real-time. In some embodiments, the exterior of the devices are constructed such that interfaces, including capillary ports, and the like, known in the art may be conveniently and reliably accommodated.

In preferred embodiments, the channels of the devices are constructed with materials and dimensions that exhibit low intrinsic fluorescence. Thus, in some embodiments, the substrate of the devices comprises fused silica as fused silica exhibits low intrinsic fluorescence. In some embodiments, the devices comprise binning channels for post-separation analysis or further manipulations. In preferred embodiments, the devices are made by subtractive processing methods known in the art that limit exposure of the native fused silica surface to only O₂ plasmas.

Devices can be constructed from a sandwich of two or more substrates so that channels are bounded conduits with ports. Bonding of the substrates may be done using methods known in the art including thermal bonding of nanofluidic channels in fused silica See e.g. Tamaki, et al. (2003) Proc. MicroTotal Analysis Systems 1:681, which is herein incorporated by reference.

CNC devices may be combined with other devices that employ separation methods known in the art including on-chip using chromatography, such as electrochromatography (Singh (2002) Anal. Chem. 74:784-789, which is herein incorporated by reference), micellar electrokinetic chromatography, or reverse-phase high pressure liquid chromatography, and the like, as well as sample preparation methods, chemical processing methods, and biological methods known in the art.

In addition to the specific embodiments shown, it is to be understood that a variety of configurations of perturbers may be used for a particular application-combining one or a plurality of perturbers and perturber types, described above, in any combination. Further, various geometries and device configurations may, according to the present invention, be readily designed by one skilled in the art for desired versatility and performance.

To the extent necessary to understand or complete the disclosure of the present invention, all publications, patents, and patent applications mentioned herein are expressly incorporated by reference therein to the same extent as though each were individually so incorporated.

Having thus described exemplary embodiments of the present invention, it should be noted by those skilled in the art that the within disclosures are exemplary only and that various other alternatives, adaptations, and modifications may be made within the scope of the present invention. Accordingly, the present invention is not limited to the specific embodiments as illustrated herein, but is only limited by the following claims.

We claim:

1. A method of manipulating an analyte in a fluid in a channel which comprises subjecting the analyte to a primary flow field and a secondary flow field produced by at least one field non-uniformity resulting from at least one perturber, with the proviso that where the perturber is a ridge obstacle or a valley obstacle, the primary flow field is not an electrokinetic field and the secondary flow field is not a dielectrophoretic field.

2. The method of claim 1, wherein the primary flow is an electrokinetic flow or a hydrodynamic flow.

3. The method of claim 1, wherein the perturber is an obstacle, a patch, or a projection.

4. The method of claim 1, wherein the perturber is elongated.

5. The method of claim 1, wherein the secondary flow field is an electrophoretic field, a dielectrophoretic field, a magnetophoretic field, an electrostriction field, a photophoretic field, a thermophoretic field, an entropic field, an acoustical field, or a chemical field.

6. The method of claim 1, wherein the secondary flow field is produced by a plurality of field non-uniformities in a coherent array.

7. The method of claim 1, which comprises placing the perturber at an angle that is substantially perpendicular to the primary flow field.

8. The method of claim 1, which comprises placing the perturber at an angle to the perpendicular of the primary flow field.

9. The method of claim 1, which comprises placing the field non-uniformity at an angle that is substantially perpendicular to the primary flow field.

10. The method of claim 1, which comprises placing the field non-uniformity at an angle to the perpendicular of the primary flow field.

11. An assay for an analyte in a fluid which comprises manipulating the analyte according to the method of claim 1, and observing or detecting the secondary flow of the analyte.

12. A microfluidic device comprising at least one perturber capable of producing at least one field non-uniformity in a primary flow field in a channel, with the proviso that where the perturber is a ridge obstacle or a valley obstacle, the primary flow field is not an electrokinetic field and the secondary flow field produced by the field non-uniformity is not a dielectrophoretic field.

13. The microfluidic device of claim 12, wherein the perturber is an obstacle, a patch, or a projection.

14. The microfluidic device of claim 12, wherein the perturber is elongated.

15. The microfluidic device of claim 12, which comprises a plurality of perturbers.

16. The microfluidic device of claim 15, wherein the perturbers of the plurality are the same.

17. The microfluidic device of claim 15, wherein the plurality comprises two or more different perturbers.

18. The microfluidic device of claim 12, which comprises two or more domains.

19. The microfluidic device of claim 12, wherein the perturber is at an angle that is substantially perpendicular to the primary flow field.

20. The microfluidic device of claim 12, wherein the perturber is at an angle to the perpendicular of the primary flow field.

21. The microfluidic device of claim 12, wherein the field non-uniformity is at an angle that is substantially perpendicular to the primary flow field.

22. The microfluidic device of claim 12, wherein the field non-uniformity is at an angle to the perpendicular of the primary flow field.
